(19) United States
(12) Patent Application Publication
(10) Pub. No.: US 2026/0076645 A1
(43) Pub. Date: Mar. 19, 2026

(54) ULTRASOUND DIAGNOSTIC APPARATUS AND ATTENUATION MAP GENERATION METHOD

(71) Applicant: FUJIFILM Corporation, Tokyo (JP)

(72) Inventors: Mika NAGAI, Tokyo (JP); Teruyuki SONOYAMA, Tokyo (JP)

(73) Assignee: FUJIFILM Corporation, Tokyo (JP)

(21) Appl. No.: 19/305,336

(22) Filed: Aug. 20, 2025

(30) Foreign Application Priority Data
Sep. 17, 2024 (JP) 2024-160415

(51) Int. Cl. A61B 8/08; A61B 8/00

(52) U.S. Cl. CPC A61B 8/085; A61B 8/469; G06T 2207/10132

(57) ABSTRACT

A brightness average map and a brightness SD map are generated based on a first data group in a first region of interest. A first extraction process using a first threshold value and a second threshold value is applied to the brightness average map, and thus a first mask is generated. A second extraction process is applied to the brightness SD map, and thus a second mask is generated. An attenuation map is generated according to a composite mask based on the first mask and the second mask.

### CROSS REFERENCE TO RELATED APPLICATION

This application claims priority to Japanese Patent Application No. 2024-160415 filed on Sep. 17, 2024 which is incorporated herein by reference in its entirety including the specification, claims, drawings, and abstract.

### BACKGROUND OF THE INVENTION

### 1. Field of the Invention

The present disclosure relates to an ultrasound diagnostic apparatus and an attenuation map generation method, and particularly to generation of a mask used in generating an attenuation map.

### 2. Description of the Related Art

The ultrasound diagnostic apparatus has an attenuation measurement function. For example, the attenuation measurement function is used in an ultrasound examination of a liver, particularly in an ultrasound examination of the liver to evaluate presence or absence of fatty liver or severity of fatty liver. Specifically, an attenuation rate reflecting characteristics of the liver is calculated based on echo data obtained from the liver, and the calculated attenuation rate is displayed as a numerical value. The attenuation rate is attenuation information and corresponds to an attenuation coefficient or an attenuation amount. In the following, the attenuation rate will be simply referred to as attenuation depending on a case. As a method of calculating the attenuation rate, various methods have been proposed in the related art.

An ultrasound diagnostic apparatus having an attenuation map display function is known. The attenuation map is, for example, a color two-dimensional map representing an attenuation rate at each position in the liver. The attenuation rate of each position can be recognized from the hue of each position in the attenuation map. Through observation of the attenuation map, a measurement region for measuring the attenuation rate as numerical information is determined, or the extent or severity of a disease in the liver is evaluated.

WO2017-068892A discloses an ultrasound diagnostic apparatus having an attenuation map display function. JP2017-158917A also discloses an ultrasound diagnostic apparatus having an attenuation map display function. In the ultrasound diagnostic apparatus disclosed in JP2017-158917A, a variance ratio (a numerical value based on a variance value and a mean value) is calculated over an entire region of interest, and a structure in the liver is specified based on the variance ratio. In JP2017-158917A, a distribution of a plurality of variance ratios or a variance ratio map substantially functions as a mask.

WO2017-068892A and JP2017-158917A do not disclose combination of a plurality of masks. WO2017-068892A and JP2017-158917A do not disclose a mask for a region other than a structure image (for example, a shadow as a weak echo region).

### SUMMARY OF THE INVENTION

A tomographic image showing a tissue such as the liver includes a region of interest and a region of non-interest. Examples of the region of non-interest include a vascular wall image, a diaphragm image, and a tumor contour image. These are generally regions with high brightness and with a large standard deviation. In addition, examples of the region of non-interest include a shadow, a blood image, and a cystic fluid image. The shadow is a low-brightness region that occurs in a deep portion of a tomographic image, or a low-brightness region that occurs at an end part or the like of the tomographic image due to poor contact between a probe and a living body surface.

In a case where the region of non-interest is expressed in color as a part of an attenuation map, it is difficult to correctly set a measurement region for accurately measuring the attenuation rate. Additionally, it is difficult to correctly evaluate characteristics of the tissue through observation of the attenuation map.

An object of the present disclosure is to exclude a region of non-interest from an attenuation map. Additionally, an object of the present disclosure is to exclude a low-brightness region, such as a shadow, from the attenuation map in addition to a structure image.

According to the present disclosure, there is provided an ultrasound diagnostic apparatus comprising: a processor, in which the processor is configured to generate an ultrasound image based on a first data set acquired by transmission and reception of first ultrasound, generate a plurality of types of masks for excluding a plurality of types of regions of non-interest from a first region of interest set in a beam scanning plane, based on the first data set, generate a composite mask by combining the plurality of types of masks, specify an attenuation display region within the first region of interest based on the composite mask, and generate an attenuation map representing an attenuation distribution in the attenuation display region based on a second data set acquired by transmission and reception of second ultrasound different from the first ultrasound.

According to the present disclosure, there is provided an attenuation map generation method executed in an information processing apparatus, the attenuation map generation method comprising: a step of generating an ultrasound image based on a first data set acquired by transmission and reception of first ultrasound; a step of generating a plurality of types of masks for excluding a plurality of types of regions of non-interest from a first region of interest set in a beam scanning plane, based on the first data set; a step of generating a composite mask by combining the plurality of types of masks; a step of specifying an attenuation display region within the first region of interest based on the composite mask; and a step of generating an attenuation map representing an attenuation distribution in the attenuation display region based on a second data set acquired by transmission and reception of second ultrasound different from the first ultrasound.

According to the present disclosure, it is possible to exclude the region of non-interest from the attenuation map. Additionally, according to the present disclosure, it is possible to exclude a low-brightness region, such as a shadow, from the attenuation map in addition to a structure image.

### BRIEF DESCRIPTION OF THE DRAWINGS

FIG. 1 is a block diagram showing a configuration example of an ultrasound diagnostic apparatus according to an embodiment.

FIG. 2 is a diagram showing a plurality of regions of interest set on a tomographic image.

FIG. 3 is a diagram showing an algorithm executed in a mask generator.

FIG. 4 is a diagram showing an example of a brightness histogram.

FIG. 5 is a diagram showing a composite mask and an attenuation map.

FIG. 6 is a diagram showing an example of an image displayed on a display.

### DESCRIPTION OF THE PREFERRED EMBODIMENTS

Hereinafter, an embodiment will be described with reference to the drawings.

### (1) Summary of Embodiment

An ultrasound diagnostic apparatus according to the embodiment includes a processor. The processor is configured to generate an ultrasound image based on a first data set acquired by transmission and reception of first ultrasound, generate a plurality of types of masks for excluding a plurality of types of regions of non-interest from a first region of interest set in a beam scanning plane, based on the first data set, generate a composite mask by combining the plurality of types of masks, specify an attenuation display region within the first region of interest based on the composite mask, and generate an attenuation map representing an attenuation distribution in the attenuation display region based on a second data set acquired by transmission and reception of second ultrasound different from the first ultrasound.

With the above-described configuration, various regions of non-interest in a living body are excluded from the attenuation map. This makes it easy to set a measurement region while avoiding the region of non-interest. Additionally, it is possible to avoid erroneous evaluation based on attenuation information in the region of non-interest. For example, in a liver, a region of interest corresponds to homogeneous liver parenchyma. The region of non-interest is, for example, an image of a contour of a structure, an image of the inside of a structure, or a shadow. Each of them is a high-brightness region or a low-brightness region in the ultrasound image.

In addition, with the above-described configuration, the plurality of types of masks are individually generated. Therefore, the individual masks can be easily generated or functions of the individual masks can be easily adjusted. Depending on the situation, the number or combination of a plurality of masks to be combined may be changed. Each mask has a function of masking the region of non-interest. A filter having a function of extracting the region of interest substantially corresponds to the mask. In a case of generating the plurality of masks, all of the first data set may be referred to, or a part of the first data set may be referred to.

In the embodiment, the processor is configured to generate a brightness average map representing a plurality of brightness averages in the first region of interest based on the first data set, and generate a first mask included in the plurality of types of masks based on the brightness average map.

In the embodiment, the processor is configured to calculate a reference value based on the first data set, calculate a first threshold value and a second threshold value different from each other based on the reference value, apply an extraction process using the first threshold value and the second threshold value to the brightness average map, and generate the first mask in which a result of the extraction process is reflected.

By setting the first threshold value and the second threshold value based on the reference value, it is possible to determine an appropriate first threshold value and an appropriate second threshold value that are suitable for the situation. Therefore, the result of the extraction process is improved. In generating the reference value, all or a part of the first data set is referred to.

In the embodiment, the processor is configured to generate the brightness average map based on a data group in the first region of interest in the first data set, and set the reference value based on a data group in a second region of interest in the first data set. The second region of interest corresponds to a portion of the first region of interest. With this configuration, it is possible to reduce the likelihood that data in the region of non-interest is referred to in a case of setting the reference value.

In the embodiment, the processor is configured to set a measurement region in the first region of interest, and calculate attenuation information as numerical information based on a data group in the measurement region in the second data set. The second region of interest is an intermediate region that is included in the first region of interest and that includes the measurement region. Shadows are likely to occur in a deep region, a right end region, and a left end region in the first region of interest. The second region of interest is determined in a region other than such a region.

In the embodiment, a depth range of the second region of interest is the same as a depth range of the measurement region. With this configuration, the calculation of the reference value is less susceptible to the influence of the structure image. A size of the second region of interest in a beam scanning direction may be set to be within a range of ¼ to ¾ of a size of the first region of interest in the beam scanning direction.

In the embodiment, the first threshold value is a threshold value for extracting a high-brightness region as a mask region. The second threshold value is a threshold value for extracting a low-brightness region as a mask region. The processor is configured to, in the extraction process, extract a plurality of brightness averages exceeding the first threshold value and a plurality of brightness averages lower than the second threshold value from the brightness average map.

In the embodiment, the processor is configured to generate a brightness variation map representing a plurality of brightness variations in the first region of interest based on the first data set, and generate a second mask included in the plurality of types of masks based on the brightness variation map. The brightness variation is, for example, a brightness standard deviation or a brightness dispersion.

In the embodiment, the processor is configured to extract a plurality of brightness variations exceeding a third threshold value from the brightness variation map to generate the second mask. In the embodiment, the second mask has a function of masking an edge of a structure image and a vicinity of the edge.

In the embodiment, a center frequency of the second ultrasound is higher than a center frequency of the first ultrasound, and a frequency band of the second ultrasound is narrower than a frequency band of the first ultrasound. With this configuration, it is possible to maintain or improve the quality of the ultrasound image, and to increase the attenuation calculation accuracy.

According to the present disclosure, there is provided an attenuation map generation method executed in an information processing apparatus, the attenuation map generation method comprising: a step of generating an ultrasound image based on a first data set acquired by transmission and reception of first ultrasound; a step of generating a plurality of types of masks for excluding a plurality of types of regions of non-interest from a first region of interest set in a beam scanning plane, based on the first data set; a step of generating a composite mask by combining the plurality of types of masks; a step of specifying an attenuation display region within the first region of interest based on the composite mask; and a step of generating an attenuation map representing an attenuation distribution in the attenuation display region based on a second data set acquired by transmission and reception of second ultrasound different from the first ultrasound.

A program for executing the above-described method is installed on the information processing apparatus via a network or a portable storage medium. The information processing apparatus is an apparatus including a processor, and is, for example, an ultrasound diagnostic apparatus. The information processing apparatus includes a storage medium that non-temporarily stores the program. The program corresponds to a program product.

### (2) Details of Embodiment

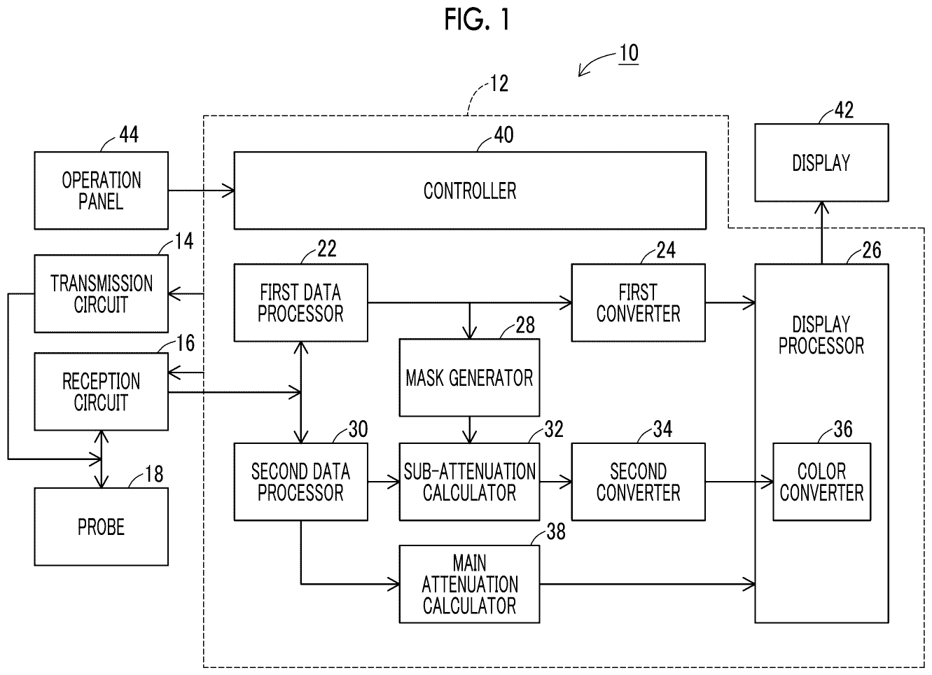

FIG. 1 shows a configuration example of the ultrasound diagnostic apparatus according to the embodiment. An ultrasound diagnostic apparatus 10 is a medical apparatus used in an ultrasound examination of a subject. Specifically, the ultrasound diagnostic apparatus 10 is used in an ultrasound examination of the liver. The ultrasound diagnostic apparatus 10 may be used in ultrasound examinations of other tissues. The ultrasound diagnostic apparatus 10 is an information processing apparatus.

The ultrasound diagnostic apparatus 10 includes a processor 12. In practice, the processor 12 is composed of one or a plurality of processing devices. Each device executes a program. The processor 12 may be configured as a CPU. In FIG. 1, a plurality of functions exhibited by the processor 12 are represented by a plurality of blocks. A transmission circuit 14, a reception circuit 16, a display 42, and an operation panel 44 are connected to the processor 12. A memory (not shown) is also connected to the processor 12. The processor 12 may have a memory.

The transmission circuit 14 and the reception circuit 16 are connected to a probe 18. The probe 18 has a transducer array consisting of a plurality of transducers. An ultrasound beam is formed by the transducer array, and electronic scanning with the ultrasound beam is performed. The electronic scanning with the ultrasound beam forms a beam scanning plane in the subject. The beam scanning plane is a two-dimensional echo data acquisition region. The probe 18 is a so-called convex type probe. Other types of probes may also be used as the probe 18. The probe 18 may be provided with a 2D transducer array.

In the embodiment, a first ultrasound beam and a second ultrasound beam different from each other are formed. The electronic scanning with the first ultrasound beam and the electronic scanning with the second ultrasound beam may be alternately repeated. The formation of the first ultrasound beam and the formation of the second ultrasound beam may be alternately repeated in association with the electronic scanning. In any case, first reception frame data is obtained for each electronic scanning with the first ultrasound beam, and second reception frame data is obtained for each electronic scanning with the second ultrasound beam.

More specifically, the first ultrasound beam is an ultrasound beam for forming a tomographic image. The first ultrasound beam (a first transmission beam and a first reception beam) is formed by the transmission and reception of first ultrasound. The second ultrasound beam is an ultrasound beam for attenuation measurement. The second ultrasound beam (a second transmission beam and a second reception beam) is formed by the transmission and reception of second ultrasound. A center frequency of the second ultrasound is higher than a center frequency of the first ultrasound. A frequency band of the second ultrasound is narrower than a frequency band of the first ultrasound.

The transmission circuit 14 supplies a plurality of transmission signals in parallel to the transducer array in the formation of the transmission beam. The reception circuit 16 is a circuit that processes a plurality of reception signals output in parallel from the transducer array in the formation of the reception beam. Specifically, the reception circuit 16 applies phasing addition to the plurality of reception signals. As a result, reception beam data is obtained. A plurality of reception beam data arranged in an electronic scanning direction constitute reception frame data.

In the configuration example shown in FIG. 1, the first reception frame data output from the reception circuit 16 is sent to a first data processor 22, and the second reception frame data output from the reception circuit 16 is sent to a second data processor 30. The first data processor 22 processes each beam data to form a tomographic image (B-mode tomographic image). The beam data processing includes processes such as envelope detection and logarithmic compression. The second data processor 30 processes each beam data prior to attenuation calculation.

In the embodiment, as will be described below, a first region of interest is set in the beam scanning plane, and a second region of interest is set in the first region of interest. In addition, a measurement region is set in the first region of interest. More specifically, the measurement region is set in the second region of interest. The beam scanning plane is formed by the electronic scanning with the first ultrasound beam. The first region of interest is a region in which with electronic scanning with the second ultrasound beam is performed. The first region of interest corresponds to the maximum range of an attenuation display region (attenuation map display region). The second region of interest corresponds to an intermediate region between the first region of interest and the measurement region. The second region of interest is a region that is referred to in the calculation of the reference value described below.

A first converter 24 applies coordinate transformation or the like to the first reception frame data output from the first data processor 22. As a result, first display frame data is generated. The first display frame data is sent to a display processor 26. In practice, a first display frame data sequence consisting of a plurality of first display frame data items aligned on a time axis is sent to the display processor 26. The first display frame data sequence corresponds to a moving image (tomographic image).

A mask generator 28 generates a plurality of types of masks based on the first reception frame data (first beam data set corresponding to the beam scanning plane) and combining the masks, and generates a composite mask by combining the plurality of types of masks. The composite mask exhibits a function of extracting the region of interest and masking the region of non-interest. The generation of the composite mask will be described in detail below. The composite mask may be generated based on the first display frame data.

A sub-attenuation calculator 32 applies attenuation calculation to a non-masked data group, that is, an extracted data group, within the second reception frame data (second beam data set corresponding to the first region of interest) output from the second data processor 30, thereby generating an attenuation map. The attenuation map represents an attenuation distribution in the attenuation display region. The individual attenuations constituting the attenuation distribution correspond to an attenuation rate, an attenuation coefficient, an attenuation amount, and the like.

A second converter 34 applies coordinate transformation or the like to the attenuation map. As will be described below, the attenuation map on an re coordinate system is transformed into the attenuation map on an xy coordinate system. The first converter 24 and the second converter 34 each correspond to a digital scan converter (DSC).

The display processor 26 includes a color converter 36. The color converter 36 generates a color attenuation map based on the attenuation map output from the second converter. The degree of the attenuation is represented by a change in hue.

A main attenuation calculator 38 calculates an attenuation rate as attenuation information numerical information based on a beam data sequence in the measurement region set in the first region of interest. The measurement region is determined based on a user's designation with reference to the attenuation map. For example, the measurement region is determined in a homogeneous region (liver parenchyma region) in the attenuation map.

The main attenuation calculator 38 calculates the attenuation information with high accuracy. With respect to this, the sub-attenuation calculator 32 simply and quickly calculates the attenuation information. An attenuation calculation expression used in the main attenuation calculator 38 and an attenuation calculation expression used in the sub-attenuation calculator 32 may be the same or different from each other. In a case where the same attenuation calculation expression is used, a reference range and a reference resolution may be different.

The display 42 is composed of, for example, an organic EL display device or a liquid crystal display. On a screen of the display 42, a monochrome tomographic image on which the color attenuation map is superimposed is displayed, and the attenuation rate as the numerical information is displayed. In addition, graphics representing a plurality of regions of interest and the like are displayed.

A controller 40 controls an operation of each component in the ultrasound diagnostic apparatus. The operation panel 44 includes a plurality of switches, a plurality of knobs, a keyboard, a trackball, and the like.

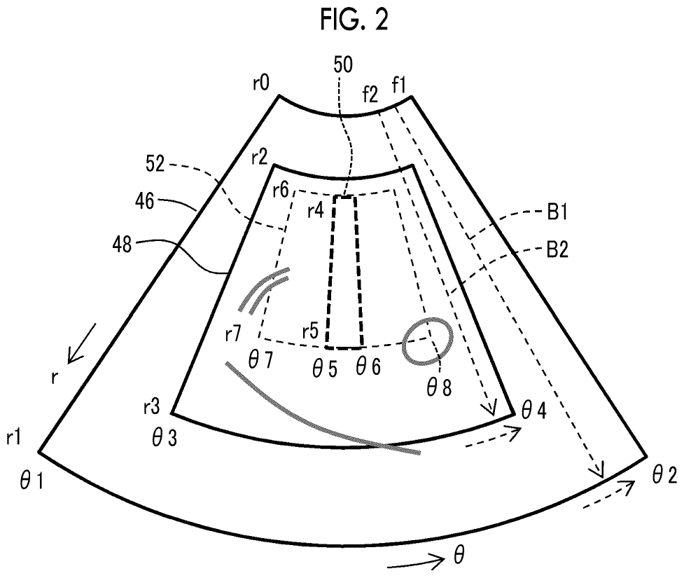

FIG. 2 shows a plurality of regions of interest. 0 indicates a beam scanning direction, and r indicates a depth direction.

A beam scanning plane 46 has a fan shape. The beam scanning plane 46 is formed by electronic scanning with a first ultrasound beam B1. A center frequency of the first ultrasound beam is denoted by f1. A range of the beam scanning direction on the beam scanning plane 46 is a range from θ1 to θ2. A range of the depth direction on the beam scanning plane 46 is a range from r0 to r1. A plurality of structures are included in the beam scanning plane 46.

A first region of interest 48 is determined in the beam scanning plane 46 based on a user's designation or automatically. The first region of interest 48 corresponds to an electronic scanning range of a second ultrasound beam B2. A center frequency of the second ultrasound beam is denoted by f2. f1<f2. A range of the beam scanning direction in the first region of interest 48 is a range from 03 to 04. A range of the depth direction in the first region of interest 48 is a range from r2 to r3.

A measurement region 50 is determined in the first region of interest 48. The measurement region 50 is a range that is referred to in a case of calculating the attenuation rate as the numerical information. A range of the beam scanning direction in the measurement region 50 is a range from 05 to 06. A range of the depth direction in the measurement region 50 is a range from r4 to r5. The measurement region 50 is a region that extends over a plurality of second ultrasound beams aligned in the electronic scanning direction.

In the embodiment, a second region of interest 52 that is referred to in a case of calculating the reference value is set in the beam scanning plane 46. The second region of interest 52 is an intermediate region that is included in the first region of interest 48 and that includes the measurement region 50. A range of the beam scanning direction in the second region of interest 52 is a range from θ7 to θ8. A range of the depth direction in the second region of interest 52 is a range from r6 to r7.

In the example shown in FIG. 2, 07 is set between θ3 and θ5, and θ8 is set between θ6 and θ4. r4 matches r6, and r5 matches r7. The illustrated second region of interest 52 is an example.

Shadows are likely to occur in a deep portion of the beam scanning plane 46, and a structure image is likely to occur in a deep portion of the tomographic image of the liver. Shadows are likely to occur in a right end portion and a left end portion of the beam scanning plane 46 due to the probe being separated from a living body surface. In addition, multiple reflections are likely to occur in a near field region of the ultrasound probe, and, in the liver, a skin layer or a fat layer appears in the near field region. In a case where the second region of interest 52 is set while avoiding such several regions of non-interest, it is possible to set an appropriate first threshold value and an appropriate second threshold value through appropriate setting of the reference value.

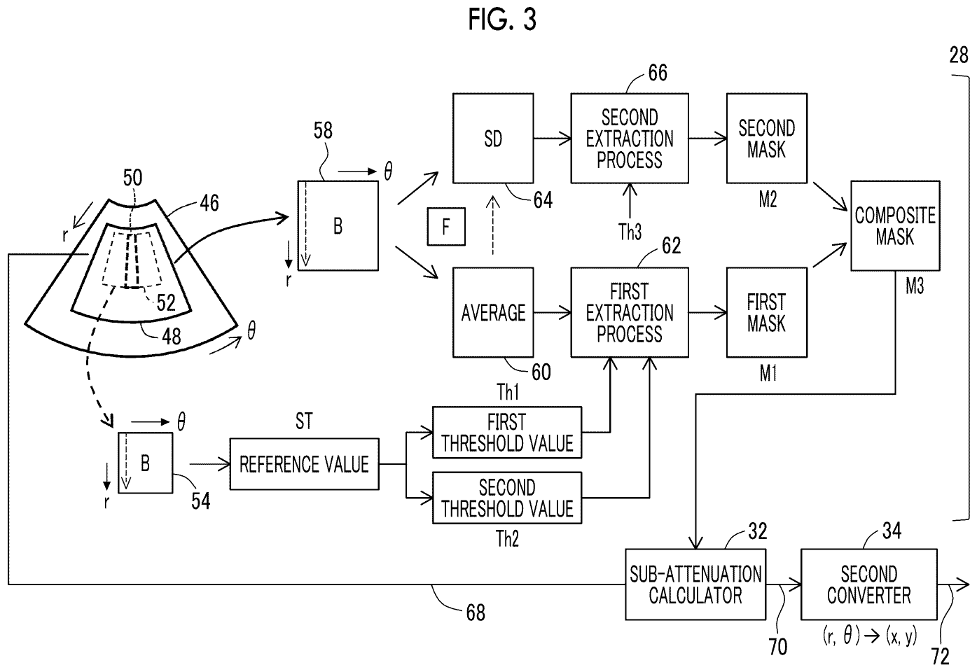

FIG. 3 shows an algorithm executed by the mask generator 28. As described above, the first region of interest 48 is set in the beam scanning plane 46, and the second region of interest 52 is set in the first region of interest 48. In addition, the measurement region 50 is set in the first region of interest 48. In order to support the setting of the measurement region 50, a masked color attenuation map is displayed according to an algorithm described below.

A first data group 58 corresponding to the first region of interest 48 is cut out from the first beam data set corresponding to the beam scanning plane 46. The first data group 58 conforms to a coordinate system defined by the electronic scanning direction θ and the depth direction r.

A brightness average map 60 and a brightness standard deviation map (brightness SD map) 64 are generated by applying a filter F as a spatial operator to the first data group 58. The filter F has a two-dimensional size centered on each coordinate, and calculates an average (mean value) and a standard deviation. The standard deviation is brightness variation information, and the variance may be calculated instead of the standard deviation. Here, the brightness corresponds to echo intensity. A size of the filter F is determined according to a size of a structure image to be detected.

On the other hand, a second data group 54 corresponding to the second region of interest 52 is cut out from the first beam data set corresponding to the beam scanning plane 46. The second data group 54 also conforms to a coordinate system defined by the electronic scanning direction θ and the depth direction r. The second data group 54 may be cut out from the first data group 58.

A reference value ST is obtained by average calculation based on the second data group 54. The second data group 54 may be pre-processed before the average calculation. A first threshold value Th1 is calculated by adding a first coefficient to the reference value ST. The first threshold value Th1 is a threshold value for masking a high-brightness structure. In addition, a second threshold value Th2 is calculated by subtracting a second coefficient from the reference value ST. The second threshold value Th2 is a threshold value for masking a low-brightness structure image (blood image, cystic fluid image, and the like) or a poor echo region, that is, a shadow.

In a first extraction process 62, the region of non-interest is extracted using the first threshold value and the second threshold value. Specifically, data having brightness exceeding the first threshold value is extracted, and, simultaneously with this, data having brightness lower than the second threshold value is extracted. A high-brightness region of non-interest and a low-brightness region of non-interest are extracted by the first extraction process 62. A first mask M1 is generated by the first extraction process 62.

In a second extraction process 66, data having a brightness variation exceeding a third threshold value is extracted from the brightness SD map 64. With the second extraction process 66, not only an edge of the structure image but also the vicinity of the edge are extracted. In the first extraction process 62, the vicinity of the edge cannot be extracted, but, in the second extraction process 66, the vicinity of the edge can be extracted. In a case where the mask is applied up to the vicinity of the edge, it is possible to visually recognize the edge of the structure image on the tomographic image without being obstructed by the color attenuation map. In addition, it is easy to determine a measurement region with a certain margin from the structure image.

A composite mask M3 is generated by combining the first mask M1 and a second mask M2, specifically, by adding the first mask M1 and the second mask M2. Three or more types of masks may be combined.

The sub-attenuation calculator 32 generates an attenuation map 70 based on non-masked data in the first data group 58 in the first region of interest. That is, the attenuation map 70 is a map avoiding the region of non-interest. In the second converter 34, the attenuation map 70 according to r0 coordinates is transformed into an attenuation map 72 according to xy coordinates. The attenuation map 72 after transformation is transformed into a color attenuation map. The color attenuation map is superimposed and displayed on a monochrome tomographic image. The measurement region 50 is determined with reference to the color attenuation map.

In the embodiment, for example, the attenuation rate may be calculated as follows. A one-dimensional attenuation sequence A is defined by Equation (1) based on a one-dimensional reception intensity sequence P(x).

$$A = - \frac{1}{2 f} ( 20 {log}_{10} ( \frac{P ( x )}{{P}_{ref} ( x )} ) ) \qquad ( 1 )$$

Here, f is an ultrasound frequency (typically, a center frequency), and x is a depth (coordinate in the depth direction). The reception intensity sequence P(x) consists of a plurality of reception intensities obtained from a plurality of observation points arranged in the depth direction in the region of interest.

In Equation (1), the reception intensity sequence P(x) is compared with a reference intensity sequence (reference signal) P_{ref} (x). The reference intensity sequence P_{ref} (x) is defined by, for example, Equation (2). The following y is a coefficient.

$${P}_{ref} ( x ) = {10}^{- γ x} \qquad ( 2 )$$

The reference intensity sequence P_{ref} (x) may be defined by other functions. The reference intensity sequence may be given as a numerical value sequence.

An attenuation rate sequence αᵣ is calculated, for example, according to Equation (3).

$${α}_{r} = \frac{1}{M} {\sum }_{i = 1}^{M} \frac{( A ( {x}_{i} ) - A ( {x}_{i - 1} ) )}{{x}_{i} - {x}_{i - 1}} \qquad ( 3 )$$

i indicates a depth number. Each attenuation rate constituting the attenuation rate sequence αᵣ is a relative attenuation rate. A large numerical value may be given to M in the main attenuation calculator, and a small value may be given to M in the sub-attenuation calculator.

According to Equation (4), an absolute attenuation rate sequence α is calculated.

$$α = k 1 * ( {α}_{r} + k 2 ) + k 3 \qquad ( 4 )$$

Here, k2 is a known attenuation rate corresponding to the reference intensity sequence P_{ref} (x). k1 and k3 are each a coefficient for adjustment. The attenuation rate a may be calculated by a method other than the method described above.

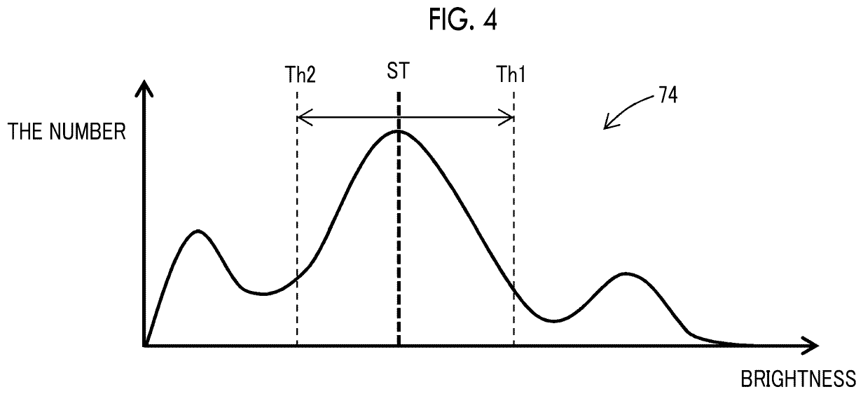

FIG. 4 shows a histogram 74 based on the second data group. A horizontal axis represents brightness (intensity), and a vertical axis represents the number. The reference value ST is determined based on the second data group, and the first threshold value Th1 and the second threshold value Th2 are determined based on the reference value ST. Since the reference value ST is adaptively determined, the first threshold value Th1 and the second threshold value Th2 that are suitable for the situation are automatically set.

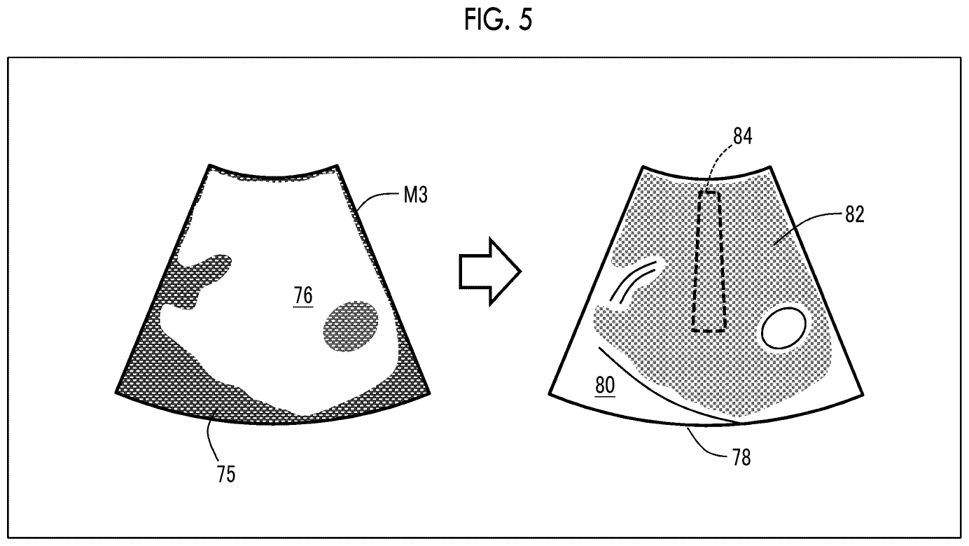

FIG. 5 shows an example of the composite mask M3, and shows an example of a color attenuation map 82. In the composite mask M3, a gray portion 75 is a masked portion, and a white portion 76 is a non-masked portion. The color attenuation map 82 is superimposed on the tomographic image. Although the tomographic image includes several structure images, the color attenuation map 82 is displayed to avoid the structure images. Reference numeral 80 indicates a portion that is not a subject of the attenuation calculation or a portion outside a color attenuation map display region. It is possible to determine a measurement region 84 at an appropriate position while referring to the color attenuation map 82.

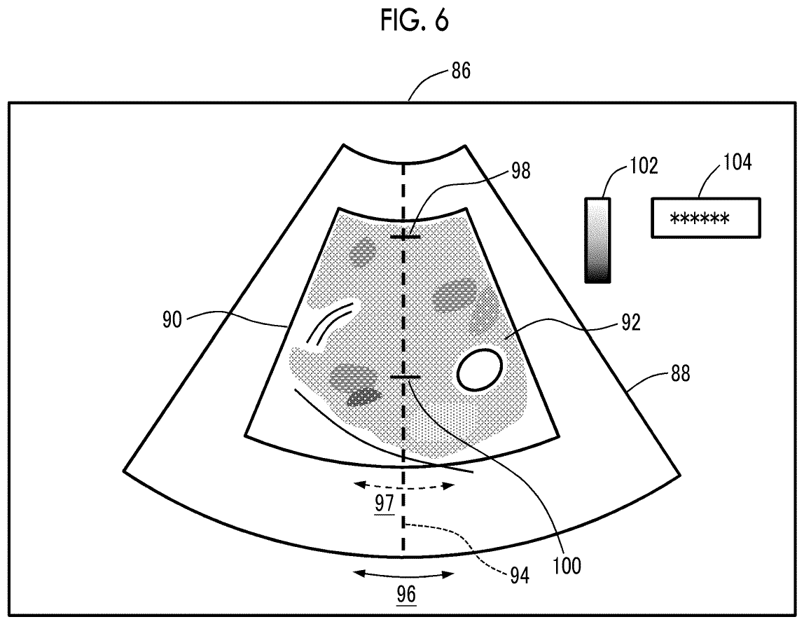

FIG. 6 shows an image 86 displayed on the display. A color attenuation map 92 is superimposed and displayed on a tomographic image 88. A graphic element 90 indicates the first region of interest. For example, the measurement region is determined by moving an azimuth cursor 94 as indicated by reference numeral 96 while referring to the color attenuation map 92. A proximal end of the measurement region is represented by a marker 98, and a distal end of the measurement region is represented by a marker 100.

As indicated by reference numeral 97, a position of the first region of interest may be changed based on the color attenuation map 92. A color bar 102 is displayed in the vicinity of the tomographic image 88. The color bar indicates a correspondence relationship between the degree of attenuation and the change in hue. Reference numeral 104 indicates an attenuation rate calculated based on the data in the measurement region.

According to the above-described embodiment, the region of non-interest can be excluded from the attenuation map. In particular, a low-brightness region such as a shadow can be excluded from the attenuation map in addition to the structure image.

What is claimed is:

1. An ultrasound diagnostic apparatus comprising:
a processor,
wherein the processor is configured to
generate an ultrasound image based on a first data set acquired by transmission and reception of first ultrasound,
generate a plurality of types of masks for excluding a plurality of types of regions of non-interest from a first region of interest set in a beam scanning plane, based on the first data set,
generate a composite mask by combining the plurality of types of masks,
specify an attenuation display region within the first region of interest based on the composite mask, and
generate an attenuation map representing an attenuation distribution in the attenuation display region based on a second data set acquired by transmission and reception of second ultrasound different from the first ultrasound.

2. The ultrasound diagnostic apparatus according to claim 1,
wherein the processor is configured to
generate a brightness average map representing a plurality of brightness averages in the first region of interest based on the first data set, and
generate a first mask included in the plurality of types of masks based on the brightness average map.

3. The ultrasound diagnostic apparatus according to claim 2,
wherein the processor is configured to
calculate a reference value based on the first data set,
calculate a first threshold value and a second threshold value different from each other based on the reference value,
apply an extraction process using the first threshold value and the second threshold value to the brightness average map, and
generate the first mask in which a result of the extraction process is reflected.

4. The ultrasound diagnostic apparatus according to claim 3,
wherein the processor is configured to
generate the brightness average map based on a data group in the first region of interest in the first data set, and
set the reference value based on a data group in a second region of interest in the first data set, and
the second region of interest corresponds to a portion of the first region of interest.

5. The ultrasound diagnostic apparatus according to claim 4,
wherein the processor is configured to
set a measurement region in the first region of interest, and
calculate attenuation information as numerical information based on a data group in the measurement region in the second data set, and
the second region of interest is an intermediate region that is included in the first region of interest and that includes the measurement region.

6. The ultrasound diagnostic apparatus according to claim 5,
wherein a depth range of the second region of interest is the same as a depth range of the measurement region.

7. The ultrasound diagnostic apparatus according to claim 3,
wherein the first threshold value is a threshold value for extracting a high-brightness region as a mask region,
the second threshold value is a threshold value for extracting a low-brightness region as a mask region, and
the processor is configured to, in the extraction process, extract a plurality of brightness averages exceeding the first threshold value and a plurality of brightness averages lower than the second threshold value from the brightness average map.

8. The ultrasound diagnostic apparatus according to claim 1,
wherein the processor is configured to
generate a brightness variation map representing a plurality of brightness variations in the first region of interest based on the first data set, and
generate a second mask included in the plurality of types of masks based on the brightness variation map.

9. The ultrasound diagnostic apparatus according to claim 8,
wherein the processor is configured to extract a plurality of brightness variations exceeding a third threshold value from the brightness variation map to generate the second mask.

10. The ultrasound diagnostic apparatus according to claim 8,
wherein the second mask has a function of masking an edge of a structure image and a vicinity of the edge.

11. The ultrasound diagnostic apparatus according to claim 1,
wherein a center frequency of the second ultrasound is higher than a center frequency of the first ultrasound, and
a frequency band of the second ultrasound is narrower than a frequency band of the first ultrasound.

12. An attenuation map generation method executed in an ultrasound diagnostic apparatus, the attenuation map generation method comprising:
a step of generating an ultrasound image based on a first data set acquired by transmission and reception of first ultrasound;
a step of generating a plurality of types of masks for excluding a plurality of types of regions of non-interest from a first region of interest set in a beam scanning plane, based on the first data set;
a step of generating a composite mask by combining the plurality of types of masks;
a step of specifying an attenuation display region within the first region of interest based on the composite mask; and
a step of generating an attenuation map representing an attenuation distribution in the attenuation display region based on a second data set acquired by transmission and reception of second ultrasound different from the first ultrasound.

13. A non-transitory storage medium storing a program for executing an attenuation map generation method in an information processing apparatus, the program causing the information processing apparatus to implement:
a function of generating an ultrasound image based on a first data set acquired by transmission and reception of first ultrasound;
a function of generating a plurality of types of masks for excluding a plurality of types of regions of non-interest from a first region of interest set in a beam scanning plane, based on the first data set;
a function of generating a composite mask by combining the plurality of types of masks;
a function of specifying an attenuation display region within the first region of interest based on the composite mask; and
a function of generating an attenuation map representing an attenuation distribution in the attenuation display region based on a second data set acquired by transmission and reception of second ultrasound different from the first ultrasound.
